(19) United States
(12) Patent Application Publication
(10) Pub. No.: US 2023/0041320 A1
(43) Pub. Date: Feb. 9, 2023

(54) Systems and Methods for Quantifying The Specific Activity of Creatininase

(71) Applicant: Polymer Technology Systems, Inc., Whitestown, IN (US)

(72) Inventor: James MURRAY, Whitestown, IN (US)

(21) Appl. No.: 17/816,945

(22) Filed: Aug. 2, 2022

Related U.S. Application Data
(60) Provisional application No. 63/203,891, filed on Aug. 3, 2021.

(51) Int. Cl. C12Q 1/34; B01L 3/00

(52) U.S. Cl. CPC C12Q 1/34; B01L 3/5023; B01L 2300/0654; B01L 2300/069; B01L 2200/16

(57) ABSTRACT

A method for determining the activity of creatininase includes providing an amount of creatininase to be measured for enzyme activity and providing an excess amount of creatinine, the excess amount being greater than an amount that will ordinarily react with the amount of creatininase. The method further includes reacting the amount of creatininase with the excess amount of creatinine to produce creatine. The method further includes reacting the creatine with diacetyl and 1-naphatol and producing a pink color. The method further includes measuring an intensity of the pink color and determining an amount of the creatine that was created based on the intensity. The method further includes calculating a specific activity of the creatininase based on the amount of creatine.

### CROSS-REFERENCE TO RELATED APPLICATIONS

This application claims the benefit of U.S. Provisional Application No. 63/203,891 filed Aug. 3, 2021, the entirety of which is hereby incorporated by reference.

### BACKGROUND

Diagnostic testing devices find usage in many scenarios, including home use, use by a doctor's office, and usage at health fairs. Diagnostic testing devices typically perform testing for various analytes in a bodily fluid and yield results that are equivalent to laboratory testing. Creatinine is a critical analyte and is an indicator of kidney function. In many scenarios, in order to measure creatinine properly, the timing of the reactions in an analysis scheme is important, which may depend on the activity of various aspects of the enzyme or other reactants.

### BRIEF SUMMARY

In one embodiment, a method for determining the activity of creatininase includes providing an amount of creatininase to be measured for enzyme activity and providing an excess amount of creatinine, the excess amount being greater than an amount that will ordinarily react with the amount of creatininase. The method further includes reacting the amount of creatininase with the excess amount of creatinine to produce creatine. The method further includes reacting the creatine with diacetyl and 1-naphatol and producing a pink color. The method further includes measuring an intensity of the pink color and determining an amount of the creatine that was created based on the intensity. The method further includes calculating a specific activity of the creatininase based on the amount of creatine. In one alternative, the intensity is measured at 520 nm. In another alternative, the creatininase is diluted in a buffer. Alternatively, the creatininase is diluted 1000-fold. Alternatively, the excess amount of creatinine is in a solution and the solution is incubated at 37° C. for 5 minutes. In another alternative, the reacting includes incubating at 37° C. for 5 minutes. Alternatively, the reacting the creatine includes providing a mixture containing 0.2% Napthtol and 0.0025% diacetyl in 0.25 M NaOH. In another alternative, the reacting the creatine includes incubating at 25° C. for 15 minutes. Alternatively, the calculating is done according to an equation:

$$Volume Activity U / mL = \frac{Δ {OD}_{520} \times {V}_{t} \times 11 \times dF}{ε \times l \times t \times Vs}$$

where ΔOD₅₂₀ is optical density measured at 520 nm, Vt is a total sample volume, 11 is a ratio of the reaction volume to a sample volume (1.1/0.1), dF is the dilution factor with respect to the enzyme, ε is an experimentally determined millimolar absorption coefficient for the pink color, l is a path length of a cuvette, t is a reaction time, and Vs is a sample volume removed from a reaction solution. Alternatively, the specific activity is calculated by multiplying the volume activity by 1/enzyme concentration (in mg/ml).

In another embodiment, a method of correcting test strip calibration includes determining an activity level of creatininase and adjusting a calibration of a test strip, based on the activity level of the creatininase. In one alternative, the calibration of the test strip is programed into a meter. In another alternative, the calibration of the test strip is stored in a storage device insertable into a meter. In one alternative, the determining the activity level includes: providing an amount of creatininase to be measured for enzyme activity and providing an excess amount of creatinine, the excess amount being greater than an amount that will ordinarily react with the amount of creatininase. The method further includes reacting the amount of creatininase with the excess amount of creatinine to produce creatine. The method further includes reacting the creatine with diacetyl and 1-naphatol and producing a pink color. The method further includes measuring an intensity of the pink color and determining an amount of the creatine that was created based on the intensity. The method further includes calculating a specific activity of the creatininase based on the amount of creatine. Alternatively, the reacting the creatine includes providing a mixture containing 0.2% Napthtol and 0.0025% diacetyl in 0.25 M NaOH. Alternatively, the calculating is done according to an equation:

$$Volume Activity U / mL = \frac{Δ {OD}_{520} ⨯ {V}_{t} ⨯ 11 ⨯ dF}{ε ⨯ l ⨯ t ⨯ Vs}$$

where ΔOD₅₂₀ is optical density measured at 520 nm, Vt is a total sample volume, 11 is a ratio of the reaction volume to a sample volume (1.1/0.1), dF is the dilution factor with respect to the enzyme, ε is an experimentally determined millimolar absorption coefficient for the pink color, l is a path length of a cuvette, t is a reaction time, and Vs is a sample volume removed from a reaction solution. Alternatively, the specific activity is calculated by multiplying the volume activity by 1/enzyme concentration (in mg/ml).

In one embodiment, a system for determining the activity of creatininase includes a reservoir of an excess amount of creatinine, the excess amount being greater than an amount that will ordinarily react with an amount of creatininase to be tested. The system further includes a vessel for reacting the amount of creatininase with the excess amount of creatinine to produce creatine. The system further includes a supply of diacetyl and 1-naphatol for reacting with the creatine to produce a pink color. The system further includes a color analyzer for analyzing the pink color and determining an intensity of the pink color. The system further includes a processor configured to determine an amount of the creatine that was created based on the intensity and calculate a specific activity of the creatininase based on the amount of creatine. In one alternative, Alternatively, the calculating is done according to an equation:

$$Volume Activity U / mL = \frac{Δ {OD}_{520} \times {V}_{t} \times 11 \times dF}{ε \times l \times t \times Vs}$$

where ΔOD₅₂₀ is optical density measured at 520 nm, Vt is a total sample volume, 11 is a ratio of the reaction volume to a sample volume (1.1/0.1), dF is the dilution factor with respect to the enzyme, ε is an experimentally determined millimolar absorption coefficient for the pink color, l is a path length of a cuvette, t is a reaction time, and Vs is a sample volume removed from a reaction solution. Alternatively, the specific activity is calculated by multiplying the volume activity by 1/enzyme concentration (in mg/ml).

### BRIEF DESCRIPTION OF THE DRAWINGS

FIG. 1 shows one embodiment of method for measuring the specific activity of creatininase;

FIG. 2 shows an embodiment of a method for measuring specific activity of creatininase and modifying calibration.

### DETAILED DESCRIPTION

Certain terminology is used herein for convenience only and is not to be taken as a limitation on the embodiments of the systems and methods for quantifying the specific activity of Creatininase. In many embodiments, the method relies on excess creatinine for reacting with the creatininase to produce creatine. This excess amount ensures that the amount of creatinine is not limiting on the speed of reaction. Furthermore, in many embodiments the creatininase is diluted which also ensures that the amount of creatinine is not limiting to the conversion process. Moreover, after creatine is created a color change is created according to many possible methods and reactants. Based on the measured color change, the amount of creatine created may be determined and this may be correlated to the specific activity of the creatininase. In some embodiments, a test strip may be further recalibrated according to a measured specific activity of creatininase. In many configurations, a standard test strip for creatinine may be created. This test strip may function somewhat differently according to the specific activity of the creatininase enzyme. Based on the specific activity measured, recalibration may be performed for a specific lot or set of test strips. This information, including but not limited to, a standard calibration curve with a specific activity correction may be stored in an electronic device such as a meter or a memory that mates with a meter, such as a MEMo Chip or other electronic storage device. A meter then performs according to the corrected calibration. Additionally, such a specific activity determination may be used to troubleshoot a set of improperly performing test strips. Additionally, in some embodiments, an automated system may be created that carries out the aspects of the analysis automatically, merely by being provided a test sample and reactants for the test sample.

Creatininase is a critical enzyme used in various analytical procedures to determine creatinine, a biomarker of kidney function. The specific activity (how much creatinine can the enzyme convert to creatine per minute) is a critical to quality attribute in many creatinine quantification assays. In many embodiments, the methods and systems provide for a means and method to quantify the specific activity of creatininase. In one embodiment, the creatininase specific activity is measured by quantifying the creatine formed by action of the enzyme. IN some embodiments, the methods and systems may provide for the extraction of creatininase activity from a dry chemistry test strip to measure residual activity. Embodiments may also be used in process quality check as well as a quality control method for product (enzyme) release testing. One feature of many embodiments, is that the method measures the reaction of interest directly, namely the specific activity of the creatininase enzyme. All other known suppliers utilize measure the activity by measuring the reverse reaction, creatine to creatinine, and using that as a surrogate measurement.

In one embodiment, creatininase is weighed out at 10 mg/mL and is diluted approximately 1000-fold in an appropriate buffer (Tris/Phosphate, possibly others). A stock solution of 100 mM creatinine is prepared in the same buffer. 1 mL of creatinine solution was heated and equilibrated at 37° C. for 5 minutes, then 100 μL of enzyme solution was added to the creatinine solution. The mixture was incubated at 37° C. for 5 minutes then a 100 μL aliquot was taken and added to 2 mL of 0.25 M NaOH. The creatine was detected by adding 1 mL of a mixture containing 0.2% Napthtol and 0.0025% diacetyl in 0.25 M NaOH. The mixture is incubated at 25° C. for 15 minutes and then transferred to a cuvette and measured at 520 nm. A blank sample is run in parallel as above where then enzyme solution is replaced with buffer. These particular concentrations and values are merely exemplary.

The creatininase activity per mL is calculated using the following equation:

$$Volume Activity U / mL = \frac{Δ {OD}_{520} \times {V}_{t} \times 11 \times dF}{ε \times l \times t \times Vs}$$

Where ΔOD520 is optical density measured at 520 nm, Vt is the total sample volume, 11 is the ratio of the reaction volume to the sample volume (1.1/0.1), dF is the dilution factor with respect to the enzyme, ε is an experimentally determined millimolar absorption coefficient for the pink-colored complex, l is the path length of the cuvette, t is the reaction time, and Vs is the sample volume removed from the reaction solution. The specific activity is calculated by multiplying the volume activity by 1/enzyme concentration (in mg/ml).

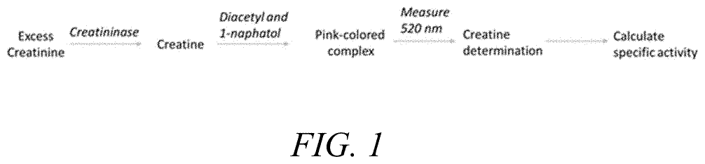

In one embodiment, as show in FIG. 1, a method is employed to calculate the specific activity of creatininase. First an excess amount of creatinine is provided. This is to ensure that creatinine does not limit the reaction rate of creatininase. Then the creatininase to be tested is added to the creatinine. In many embodiments, this will be done in an aqueous mixture. In many embodiments, the creatininase will be diluted in water or another solvent. This helps ensure that the ability of the creatininase to find unreacted creatinine is not limited. This procedure is conducted generally for a set period of time, since a point of the analysis is to determine the specific activity, which relates to how much activity the enzyme has over a period of time (in other words, how much analyte it converts over a period of time). Subsequently, Diacetyl and 1-naphatol are added to the creatine created previously. This causes a pink color to be produced. This pink colored complex is subsequently measured at 520 nm. From this, the amount of creatine may be determined. Once the creatine produced is determined, the specific activity of the enzyme may be calculated.

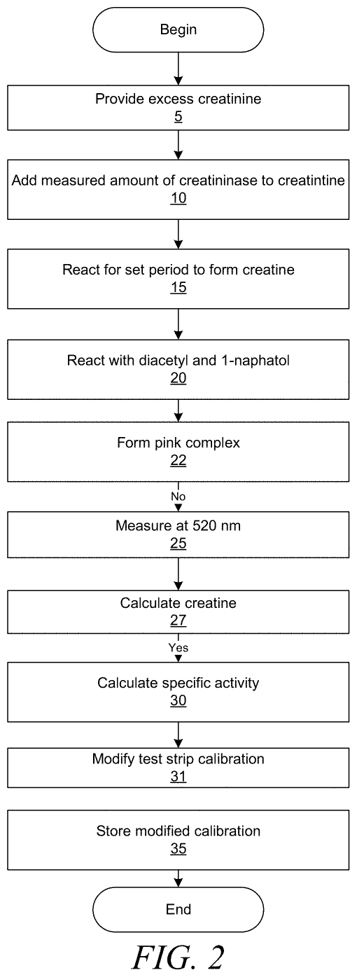

FIG. 2 shows an embodiment of a test strip calibration method. Typically, a test strip and meter is used for point of care testing of blood, urine, or other fluid sample. The sample is applied to the test strip either before or after the test strip is inserted into the meter. A reaction occurs in the test strip, in this case for the detection of creatinine, however there may be other possibilities. The meter then detects a change in the test strip and calculates the corresponding level of analyte. In many configurations this is performed via an optical system that detects a color change however, electrochemical or other detection systems are possible (fluorescence). Typically, a calibration curve is stored in the meter or on a storage medium that is inserted into the meter (MEMochip), that provides for a determination of the level of analytes. Of course, the calculation relies on previous tests which are used to create the curve. However, over time, the strength of the enzyme activity may reduce. This may be calculated into the calibration curve, which may, based on a calculated age of the test strip, automatically adjust the curve according to reduced enzyme specific activity. Additionally, a remote lab may periodically evaluate reductions in enzyme activity over time or based on differences in lots, suppliers, or other sources. These changes in specific activity may be programed into meters or storages mediums or may be electronically transmitted to meters. Additionally, such techniques may be employed in the lab to adjust test strip calibration or merely troubleshoot issues with reliability of test strips. Therefore, in the method of FIG. 2, in step 5, excess creatinine is provided. In step 10, the amount of creatininase to be reacted is provided, which may be diluted and mixed with a solution as previously explained. In step 15, the two are reacted for a period of time to produce creatine. Then, in step 20 the creatine is reacted with diacetyl and 1-naphatol to form a pink complex in step 22. In step 25, the pink complex is measured at 520 nm. Based on this measurement, the amount of creatine is calculated in step 27. In step 30 the specific activity of the creatininase is calculated. In step 31 the test strip calibration is modified. This is typically stored in a storage medium or a meter in step 35. Or instead the specific activity can simple be used to troubleshoot test strip issues.

In many embodiments, parts of the system are provided in devices including microprocessors. Various embodiments of the systems and methods described herein may be implemented fully or partially in software and/or firmware. This software and/or firmware may take the form of instructions contained in or on a non-transitory computer-readable storage medium. Those instructions then may be read and executed by one or more processors to enable performance of the operations described herein. The instructions may be in any suitable form such as, but not limited to, source code, compiled code, interpreted code, executable code, static code, dynamic code, and the like. Such a computer-readable medium may include any tangible non-transitory medium for storing information in a form readable by one or more computers such as, but not limited to, read only memory (ROM); random access memory (RAM); magnetic disk storage media; optical storage media; a flash memory, etc.

Embodiments of the systems and methods described herein may be implemented in a variety of systems including, but not limited to, smartphones, tablets, laptops, and combinations of computing devices and cloud computing resources. For instance, portions of the operations may occur in one device, and other operations may occur at a remote location, such as a remote server or servers. For instance, the collection of the data may occur at a smartphone, and the data analysis may occur at a server or in a cloud computing resource. Any single computing device or combination of computing devices may execute the methods described.

In various instances, parts of the method may be implemented in modules, subroutines, or other computing structures. In many embodiments, the method and software embodying the method may be recorded on a fixed tangible medium.

While specific embodiments have been described in detail in the foregoing detailed description, it will be appreciated by those skilled in the art that various modifications and alternatives to those details could be developed in light of the overall teachings of the disclosure and the broad inventive concepts thereof. It is understood, therefore, that the scope of this disclosure is not limited to the particular examples and implementations disclosed herein but is intended to cover modifications within the spirit and scope thereof as defined by the appended claims and any and all equivalents thereof.

1. A method for determining the activity of creatininase, the method comprising:
providing an amount of creatininase to be measured for enzyme activity;
providing an excess amount of creatinine, the excess amount being greater than an amount that will ordinarily react with the amount of creatininase;
reacting the amount of creatinianse with the excess amount of creatinine to produce creatine;
reacting the creatine with diacetyl and 1-naphatol;
producing a pink color;
measuring an intensity of the pink color;
determining an amount of the creatine that was created based on the intensity;
calculating a specific activity of the creatinianse based on the amount of creatine.

2. The method of claim 1, wherein the intensity is measured at 520 nm.

3. The method of claim 2, the creatininase is diluted in a buffer.

4. The method of claim 3, wherein the creatininase is diluted 1000-fold.

5. The method of claim 4, wherein the excess amount of creatinine is in a solution and the solution is incubated at 37° C. for 5 minutes.

6. The method of claim 5, wherein the reacting includes incubating at 37° C. for 5 minutes.

7. The method of claim 6, wherein the reacting the creatine includes providing a mixture containing 0.2% Napthtol and 0.0025% diacetyl in 0.25 M NaOH.

8. The method of claim 7, wherein the reacting the creatine includes incubating at 25° C. for 15 minutes.

9. The method of claim 8, wherein the calculating is done according to an equation:
$$Volume Activity U / mL = \frac{Δ {OD}_{520} \times {V}_{t} \times 11 \times dF}{ε \times l \times t \times Vs}$$
where ΔOD₅₂₀ is optical density measured at 520 nm, Vt is a total sample volume, 11 is a ratio of the reaction volume to a sample volume (1.1/0.1), dF is the dilution factor with respect to the enzyme, ε is an experimentally determined millimolar absorption coefficient for the pink color, l is a path length of a cuvette, t is a reaction time, and Vs is a sample volume removed from a reaction solution.

10. The method of claim 9, wherein the specific activity is calculated by multiplying the volume activity by 1/enzyme concentration (in mg/ml).

11. A method of correcting test strip calibration, the method comprising:
determining an activity level of creatininase;
adjusting a calibration of a test strip, based on the activity level of the creatininase.

12. The method of claim 11, wherein the calibration of the test strip is programed into a meter.

13. The method of claim 11, wherein the calibration of the test strip is stored in a storage device insertable into a meter.

14. The method of claim 13, wherein the determining the activity level includes:
providing an amount of creatininase to be measured for enzyme activity;
providing an excess amount of creatinine, the excess amount being greater than an amount that will ordinarily react with the amount of creatininase;
reacting the amount of creatininase with the excess amount of creatinine to produce creatine;
reacting the creatine with diacetyl and 1-naphatol;
producing a pink color;
measuring an intensity of the pink color;
determining an amount of the creatine that was created based on the intensity;
calculating a specific activity of the creatininase based on the amount of creatine.

15. The method of claim 14, wherein the reacting the creatine includes providing a mixture containing 0.2% Napthtol and 0.0025% diacetyl in 0.25 M NaOH.

16. The method of claim 15, wherein the reacting the creatine includes incubating at 25° C. for 15 minutes.

17. The method of claim 16, wherein the calculating is done according to an equation:
$$Volume Activity U / mL = \frac{Δ {OD}_{520} \times {V}_{t} \times 11 \times dF}{ε \times l \times t \times Vs}$$
where ΔOD₅₂₀ is optical density measured at 520 nm, Vt is a total sample volume, 11 is a ratio of the reaction volume to a sample volume (1.1/0.1), dF is the dilution factor with respect to the enzyme, ε is an experimentally determined millimolar absorption coefficient for the pink color, l is a path length of a cuvette, t is a reaction time, and Vs is a sample volume removed from a reaction solution.

18. The method of claim 17, wherein the specific activity is calculated by multiplying the volume activity by 1/enzyme concentration (in mg/ml).

19. A system for determining the activity of creatininase, the system comprising:
a reservoir of an excess amount of creatinine, the excess amount being greater than an amount that will ordinarily react with an amount of creatininase to be tested;
a vessel for reacting the amount of creatininase with the excess amount of creatinine to produce creatine;
a supply of diacetyl and 1-naphatol for reacting with the creatine to produce a pink color;
a color analyzer for analyzing the pink color and determining an intensity of the pink color;
a processor configured to determine an amount of the creatine that was created based on the intensity and calculate a specific activity of the creatininase based on the amount of creatine.

20. The system of claim 19, wherein the calculating is done according to an equation:
$$Volume Activity U / mL = \frac{Δ {OD}_{520} \times {V}_{t} \times 11 \times dF}{ε \times l \times t \times Vs}$$
where ΔOD₅₂₀ is optical density measured at 520 nm, Vt is a total sample volume, 11 is a ratio of the reaction volume to a sample volume (1.1/0.1), dF is the dilution factor with respect to the enzyme, ε is an experimentally determined millimolar absorption coefficient for the pink color, l is a path length of a cuvette, t is a reaction time, and Vs is a sample volume removed from a reaction solution.
